(19) United States
(12) Patent Application Publication
(10) Pub. No.: US 2025/0229055 A1
(43) Pub. Date: Jul. 17, 2025

(54) METHOD AND APPARATUS FOR TREATING SLEEP DISORDER USING DATA-DRIVEN PERSONALIZED SLEEP TIME RECOMMENDATION AND ADAPTIVE INTERVENTION CONTROL

(71) Applicant: WELT Corp., Ltd, Seoul (KR)

(72) Inventors: Sung Ji KANG, Seoul (KR); Hye Gang ROH, Seoul (KR); Hwa Young JUNG, Gyeonggi-do (KR); Danny Onsung LEE, Seoul (KR); Hye Ryung KIM, Gyeonggi-do (KR)

(21) Appl. No.: 19/170,268

(22) Filed: Apr. 4, 2025

Related U.S. Application Data
(63) Continuation-in-part of application No. 17/656,034, filed on Mar. 23, 2022.

(30) Foreign Application Priority Data
Oct. 22, 2021 (KR) 10-2021-0141738

(51) Int. Cl. A61M 21/02; A61M 21/00

(52) U.S. Cl. CPC A61M 21/02; A61M 2021/0022; A61M 2021/0066; A61M 2021/0083; A61M 2205/3303; A61M 2230/10; A61M 2230/42

(57) ABSTRACT

Provided is a method of treating a sleep disorder based on data and an apparatus for performing the method. The method of treating a sleep disorder based on data includes determining, by a sleep disorder treatment apparatus, sleep quality; determining, by the sleep disorder treatment apparatus, user compliance with a first recommended time in bed (R-TIB); and determining, by the sleep disorder treatment apparatus, a second R-TIB on the basis of the sleep quality and the user compliance, wherein the first R-TIB is a sleep time recommended to the user earlier, and the second R-TIB is a sleep time recommended to the user later. The sleep disorder treatment apparatus further controls at least one predetermined supplemental device, including a temperature controller and a haptic actuator, in real time based on the second R-TIB to promote adherence and improve therapeutic outcomes.

### CROSS-REFERENCE TO RELATED APPLICATION

This is a continuation-in-part application of the U.S. Non-Provisional patent application Ser. No. 17/656,034, filed on Mar. 23, 2022, the disclosure of which is incorporated herein by reference in its entirety.

### BACKGROUND

### 1. Field of the Invention

The present invention relates to a method of treating a sleep disorder based on data and an apparatus for performing the method. More particularly, the present invention relates to a method of treating a sleep disorder by determining a recommended time in bed (R-TIB) based on sleep efficiency and sleep deficit, and an apparatus for performing the method. The method involves analyzing user sleep data, processing missing sleep data, and determining user compliance to optimize sleep treatment outcomes.

### 2. Discussion of Related Art

With the development of various smart technologies, data regarding personal daily activities is recorded and personal lives are managed more efficiently based on the recorded data. Above all, as interest in health increases, health-related data logging is getting attention. Many users are generating and utilizing various types of data related to the health, such as exercise, diet, and sleep of users, through user devices, such as smartphones and wearable devices. That is, users have started to generate and manage their own health-related data through user devices, such as smartphones or wearable devices, departing from health-related data being generated and managed only by medical institutions in the past.

Health-related data logging is generally performed through wearable devices. A wearable device is a user device carried by a user or attached to a user's body. With the development of the Internet of Things, wearable devices are being widely used to collect health-related data. A wearable device can collect body change information of a user and data on the environment surrounding a user through the device, and can provide advice required for the health of the user based on the collected data.

Currently, a procedure of providing feedback using health-related data acquired through a wearable device is not sophisticated, and thus is not utilized for detailed medical practices. However, according to the development of not only wearable devices but also user devices capable of collecting various types of health-related data, and the refinement of decision algorithms based on health-related data acquired through user devices, health-related data acquired through user devices can be used in actual medical practices.

In particular, performing a behavioral treatment based on sleep data is an area where it can be implemented based on an algorithm. By analyzing sleep data, interactive treatment methods can be used to help improve sleep patterns and compliance. Recent approaches include real-time adaptive interventions, where environmental factors and user feedback are integrated into the treatment process. More personalized and effective digital treatment is being studied and developed in detail.

### SUMMARY OF THE INVENTION

The present invention is directed to providing a method of treating a sleep disorder based on data and a wearable sensor for performing the method that is capable of treating a sleep disorder of a user by recommending a more therapeutically effective bedtime in consideration of the sleep efficiency, sleep deficit, and real-time user compliance. These parameters are determined using objective evaluation data, including at least one of respiration data and brain wave data, measured by wearable sensor, and processed through a decision tree algorithm.”

Furthermore, the present invention provides an interactive treatment approach that incorporates predetermined supplemental device to enhance adherence to the recommended bedtime. The device actively adjusts environmental conditions through a temperature controller, which regulates sleep-conducive parameters such as ambient temperature to promote sleep onset and maintain sleep continuity. Moreover, a haptic actuator provides tactile stimulation to reinforce bedtime adherence and facilitate wakefulness at the adjusted R-TIB. These automated interventions dynamically respond to real-time sleep data, improving user compliance and optimizing therapeutic outcomes.

The present invention is also directed to providing a method of treating a sleep disorder based on data and a wearable sensor for performing the method that is capable of treating a sleep disorder of a user by appropriately adjusting a recommended bedtime of the user to control the balance between the patient's compliance and therapeutic performance using a decision tree algorithm.

The present invention is also directed to providing a method of treating a sleep disorder based on data and a wearable sensor for performing the method that are capable of treating a sleep disorder of a user through adaptive management of sleep data and generation of a recommended time in bed (R-TIB) based on a decision tree algorithm in consideration of the user compliance of the user.

The present invention is also directed to treat insomnia by preventing the user or wearer from staying awake in bed, normalizing a sleep pattern, and strengthening a connection between sleep and bed.

The technical objectives of the present invention are not limited to the above, and other objectives may become apparent to those of ordinary skill in the art based on the following descriptions.

Representative configurations of the present invention for achieving the above object are as follows.

According to an aspect of the present invention, there is provided a method of treating a sleep disorder based on data, the method including: determining, by a sleep disorder treatment apparatus, sleep quality using a decision tree algorithm; determining, by the sleep disorder treatment apparatus, user compliance with a first R-TIB using a decision tree algorithm; and determining, by the sleep disorder treatment apparatus, a second R-TIB using a decision tree algorithm on the basis of the sleep quality and the user compliance, wherein the first R-TIB is a sleep time recommended to a user earlier, and the second R-TIB is a sleep time recommended to the user later.

The sleep quality may include sleep efficiency and a sleep deficit, and the user compliance may be determined based on slopes that are set to be different according to ranges of a TIB or determined based on a user compliance curve using a piecewise function including a minimum TIB, a maximum TIB and the first R-TIB which is less than one half of the maximum TIB, the user compliance curve being parabolic.

The second R-TIB may be determined based on a user sleep efficiency range, a user sleep deficit range, the user compliance, and a difference in size between the first R-TIB and the TIB, the user sleep efficiency range may be a sleep efficiency range of the user among “a” (here, “a” is a natural number) set sleep efficiency ranges based on a first determination on the sleep efficiency (a sleep efficiency determination), the user sleep deficit range may be a user sleep deficit range of the user among “b” (here, “b” is a natural number) set sleep deficit ranges based on a second determination on the sleep deficit (a sleep deficit determination), the user sleep deficit range may be determined, after determination of the user sleep efficiency range, based on the determined user sleep efficiency range, and the second R-TIB may be determined based on a default R-TIB window or an increase R-TIB window determined in consideration of a sleep fluctuation range, wherein the increase R-TIB window may be provided such that the sleep fluctuation range is less than a threshold fluctuation range.

According to another aspect of the present invention, there is provided a sleep disorder treatment apparatus for performing a sleep disorder treatment based on data, the sleep disorder treatment apparatus including: a sleep quality determiner configured to determine sleep quality; a user compliance determiner configured to determine user compliance with a first R-TIB; and an R-TIB determiner configured to determine a second R-TIB on the basis of the sleep quality and the user compliance, wherein the first R-TIB is a sleep time recommended to a user earlier, and the second R-TIB is a sleep time recommended to the user later.

The sleep quality may include sleep efficiency and a sleep deficit using the decision tree algorithm and objective evaluation data including at least one of respiration data and brain waves data received by at least one sensor of the wearable device. The user compliance may be determined based on slopes that are set to be different according to ranges of a time in bed (TIB) or determined based on a user compliance curve using a piecewise function including a minimum TIB, a maximum TIB and the first R-TIB which is less than one half of the maximum TIB, the user compliance curve being parabolic.

An R-TIB determiner is configured to: set an R-TIB window, wherein the R-TIB window is initially set to a default R-TIB window, and a fluctuation range of the default R-TIB window is calculated based on a standard deviation, and wherein when the fluctuation range of the default R-TIB window is greater than or equal to a threshold fluctuation range, the R-TIB window is increased by adding one more day to the default R-TIB window, and wherein the R-TIB window is determined based on unmodifiable sleep data of the most recent n days; process missing sleep data; determine a first recommended time in bed (R-TIB) based on the sleep efficiency, the sleep deficit, the R-TIB window and the processed missing sleep data; and determine a second R-TIB, using the decision tree algorithm, on the basis of the sleep quality, the processed missing sleep data, and user compliance, wherein the first R-TIB is a sleep time recommended to a user earlier, and the second R-TIB is a sleep time recommended to a user later.

The second R-TIB may be determined based on a user sleep efficiency range, a user sleep deficit range, the user compliance, and a difference in size between the first R-TIB and the TIB, the user sleep efficiency range may be a sleep efficiency range of the user among “a” (here, “a” is a natural number) set sleep efficiency ranges based on a first determination on the sleep efficiency (a sleep efficiency determination), the user sleep deficit range may be a user sleep deficit range of the user among “b” (here, “b” is a natural number) set sleep deficit ranges based on a second determination on the sleep deficit (a sleep deficit determination), the user sleep deficit range may be determined, after determination of the user sleep efficiency range, based on the determined user sleep efficiency range, and the second R-TIB may be determined based on a default R-TIB window or an increase R-TIB window determined in consideration of a sleep fluctuation range, wherein the increase R-TIB window may be provided such that the sleep fluctuation range is less than a threshold fluctuation range.

The sleep disorder treatment apparatus transmits a control signal to at least one predetermined supplemental device based on the second R-TIB, and wherein the processor is configured to actively control at least one predetermined supplemental device including a temperature controller and a haptic actuator, wherein: the temperature controller is configured to induce sleep by adjusting the temperature to maintain an optimal sleep environment based on the second R-TIB, and the haptic actuator is configured to wake the user by providing stimulation based on the second R-TIB.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a conceptual diagram illustrating an apparatus for treating a sleep disorder according to an embodiment of the present invention;

FIGS. 2A and 2B are conceptual diagrams illustrating an operation of a user compliance determiner according to an embodiment of the present invention;

FIG. 3 is a conceptual diagram illustrating an operation of a recommended time in bed (R-TIB) determiner according to an embodiment of the present invention;

FIG. 4 is a conceptual diagram illustrating a method of extracting data for determining an R-TIB according to an embodiment of the present invention;

FIG. 5 is a conceptual diagram illustrating a method of processing missing data according to an embodiment of the present invention; and

FIG. 6 is a conceptual diagram illustrating a method of determining an R-TIB according to an embodiment of the present invention.

### DETAILED DESCRIPTION OF EXEMPLARY EMBODIMENTS

In the following detailed description, reference is made to the accompanying drawings that show, by way of illustration, specific embodiments in which the invention may be practiced. These embodiments are described in sufficient detail to enable those skilled in the art to practice the invention. It is to be understood that the various embodiments of the invention, although different, are not necessarily mutually exclusive. For example, a certain feature, structure, or characteristic described herein in connection with one embodiment may be implemented within other embodiments without departing from the spirit and scope of the invention. In addition, it is to be understood that the location or arrangement of individual elements within each disclosed embodiment may be modified without departing from the spirit and scope of the invention. The following detailed description is, therefore, not to be taken in a limiting sense, and the scope of the present invention is defined only by the appended claims, appropriately interpreted, along with the full range of equivalents to which the claims are entitled. In the drawings, like numerals refer to the same or similar functionality throughout the several views.

This specification may use the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub programs, or portions of code. A computer program can be deployed to be executed on one computer or on multiple computers that are located at one site or distributed across multiple sites and interconnected by a data communication network.

In this specification, the term “database” may be used broadly to refer to any collection of data: the data does not need to be structured in any particular way, or structured at all, and it can be stored on storage devices in one or more locations. Thus, for example, the index database can include multiple collections of data, each of which may be organized and accessed differently.

Similarly, in this specification the term “engine” or “determiner” may be used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, a Microsoft Cognitive Toolkit framework, an Apache Singa framework, an Apache MXNet framework, or etc.

Embodiments of the subject matter described in this specification can be implemented in a computing system that includes a back end component, e.g., as a data server, or that includes a middleware component, e.g., an application server, or that includes a front end component, e.g., a client computer having a graphical user interface, a web browser, or an app through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

Hereinafter, various exemplary embodiments of the present invention will be described in detail with reference to the accompanying drawings in order to enable those skilled in the art to easily practice the present invention.

Sleep restriction therapy is a treatment technique belonging to cognitive behavioral therapy for insomnia. Sleep restriction therapy is a behavioral treatment of limiting the time in bed (TIB). Because sleep cannot be forcefully restricted, sleep restriction therapy is performed by recommending a time in bed, and the term recommended TIB (hereinafter, R-TIB) is used for this time in bed.

Sleep restriction therapy may help treat insomnia by 1) preventing staying awake in bed, 2) normalizing a sleep pattern, and 3) strengthening a connection between sleep and bed.

Research on sleep restriction therapy conducted up to date is quite limited. Although sleep restriction therapy in its initial version has been applied to cognitive behavioral treatment for insomnia, there are still many studies in progress, such as the operation mechanisms of sleep restriction therapy and the specific methods of sleep restriction therapy.

According to an apparatus for performing the method according to an embodiment of the present invention, an advanced R-TIB algorithm based on sleep efficiency is disclosed. The aim of the advanced R-TIB algorithm is to determine a more therapeutically effective R-TIB based on various pieces of information, such as user information and environmental information, to control the balance between user compliance and therapeutic performance.

Furthermore, according to an apparatus for performing the method according to an embodiment of the present invention, wearable sensors may collect objective sleep data (e.g., respiration, brain waves) and actively controls supplemental devices such as a temperature controller and a haptic actuator to promote adherence to the recommended R-TIB.

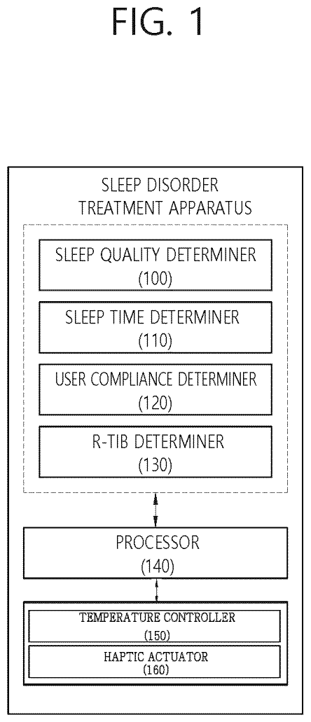

FIG. 1 is a conceptual diagram illustrating an apparatus for treating a sleep disorder according to an embodiment of the present invention.

In FIG. 1, a method of adaptively providing a user with an R-TIB for treatment of a user's sleep disorder is disclosed.

Referring to FIG. 1, a sleep disorder treatment apparatus may include a sleep quality determiner 100, a sleep time determiner 110, a user compliance determiner 120, a recommended time in bed (R-TIB) determiner 130, a processor 140, a temperature controller 150, and a haptic actuator 160.

The sleep quality determiner 100 may be implemented to determine sleep quality, including sleep efficiency and sleep deficit. The sleep quality involves whether a user has a good sleep state. The sleep quality determiner 100 may determine the sleep quality based on subjective evaluation data and/or objective evaluation data (respiration, brain waves, movement, etc.) received by at least one sensor of the wearable device.

The sleep quality determiner 100 may be implemented to perform a numerical determination on sleep efficiency and whether there is a sleep deficit, to determine a section corresponding to the user in a first determination section (a sleep efficiency determination section) and a second determination section (a sleep deficit determination) for determining an R-TIB, which will be described below.

The sleep time determiner 110 may be implemented to determine an actual sleep time (TIB) of the user. The sleep time may be a time that the user has slept in bed and may be determined based on subjective evaluation data and/or objective evaluation data (respiration, brain waves, movement, etc.) obtained from wearable sensors.

The user compliance determiner 120 may be the degree to which the user adheres to a previously recommended first R-TIB. The user compliance may also be referred to as R-TIB compliance in another expression. Based on the relationship between an R-TIB determined by the R-TIB determiner 130 and a TIB, the degree to which the user complies with the determined R-TIB may be determined as the user compliance. In the present invention, the user compliance may be determined based on the relationship between the first R-TIB and the actual TIB, using either a linear slope model or a piecewise parabolic user compliance curve, as further described below.

The R-TIB determiner 130 may be implemented to determine an R-TIB. The R-TIB may be determined in consideration of the sleep quality of the user, the actual sleep time (TIB) of the user, and the user compliance.

The R-TIB determiner 130 may be implemented to determine an R-TIB. R-TIB determiner 130 may further generate a second R-TIB based on one or more of the following: sleep quality (e.g., sleep efficiency range and sleep deficit range), actual TIB, processed missing data, and user compliance. The second R-TIB is dynamically determined using a decision tree algorithm and may involve adjustment of an R-TIB window based on sleep fluctuation range.

The processor 140 may be implemented to control the operations of the sleep quality determiner 100, the sleep time determiner 110, the user compliance determiner 120, and the R-TIB determiner 130. In addition, the processor transmits a control signal to at least one predetermined supplemental device, including a temperature controller 150 and a haptic actuator 160, based on the second R-TIB.

The temperature controller 150 is configured to induce or maintain sleep by adjusting environmental parameters such as ambient temperature in real-time. For example, it may lower the temperature to promote sleep onset or increase it to assist with waking.

The haptic actuator 160 is configured to provide tactile stimulation, such as vibration or gentle pulses, to reinforce adherence to bedtime or to help the user wake up at the second R-TIB.

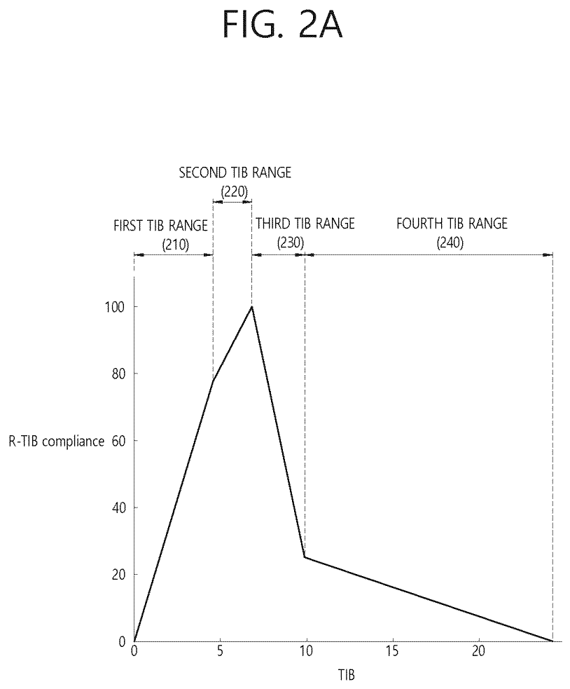

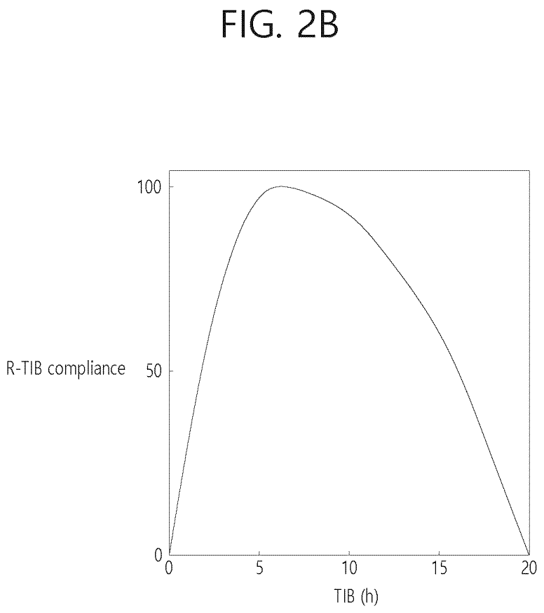

FIG. 2A is a conceptual diagram illustrating an operation of a user compliance determiner according to an embodiment of the present invention.

In FIG. 2A, a method of the user compliance determiner 120 determining user compliance is disclosed.

Referring to FIG. 2A, the user compliance may be determined using a decision three algorithm based on sleep quality, processed missing sleep data, and real-time intervention control.

Referring to FIG. 2A, the user compliance may be determined to have a different slope value assigned to different ranges of TIB. A method of determining the user compliance based on the different slope according to the range of the TIB is disclosed below.

The user compliance value may be adjusted based on the influence of real-time interventions, such as control signals transmitted to a temperature controller and a haptic actuator, which ensure adherence to the R-TIB. The ranges of the TIB and the slope values are exemplary values and may be adaptively changed based on a feedback result.

The user compliance may also vary depending on real-time evaluation of sleep quality. If sleep quality is poor, the temperature controller may be activated to induce sleep. If user significantly exceeds R-TIB, a haptic actuator may be triggered to promote wakefulness.

Furthermore, the decision tree algorithm accounts for missing sleep data. When user compliance is high, missing data is ignored. If compliance is medium, missing data is estimated using the decision tree algorithm. Lastly, if compliance is low, missing data is excluded from the calculation.

For example, the user compliance (R-TIB compliance) may be calculated as in Equation 1 below.

$$y = \{ 0 < x < 4.5 : \frac{( 100 - {s}_{1} ( a - 4.5 ) )}{4.5} x \qquad [ Equation 1 ‐ 1 ]$$

$$4.5 \leq x \leq a : {s}_{1} x + 100 - {s}_{1} a$$

$$a < x \leq a + 3 : {s}_{2} x + 100 - {s}_{2} a$$

$$a + 3 < x < M : \frac{( - 3 {s}_{2} - 100 )}{M - a - 3} x + M * \frac{( 3 {s}_{2} + 100 )}{M - a - 3} \}$$

Variables in Equation 1-1 denote the following.

y=R-TIB compliance (user compliance)

x=TIB (an actual sleep time of the user after determination of an R-TIB)

a=R-TIB (a recommended sleep time recommended to the user through the R-TIB determiner)

M=Max TIB duration (the maximum time the user can lie in bed)

s1=slope 1 for calculating the R-TIB compliance (the user compliance)

s2=slope 2 for calculating the R-TIB compliance (the user compliance)

Here, the numeric values, such as M (e.g., 24), s1, s2, and a+3 (3 is one of variables), may be adaptively changed according to a user compliance feedback result, and a treatment effect.

Specifically, a first TIB range 210 and a second TIB range 220 may be set until the TIB matches the R-TIB without exceeding the R-TIB such that the user compliance in the first TIB range 210 increases relatively rapidly with a larger slope than that in the second TIB range 220.

When the TIB exceeds the R-TIB, a third TIB range 230 and a fourth TIB range 240 may be set such that the user compliance in the third TIB range 230 decreases relatively slowly with a larger slope than that in the fourth TIB range 240.

FIG. 2B is a conceptual diagram illustrating an operation of the user compliance determiner according to the embodiment of the present invention.

In FIG. 2B, a method of the user compliance determiner 120 determining the user compliance is disclosed.

Referring to FIG. 2B, the user compliance according to the TIB may be determined based on a user compliance curve which is not linear but parabolic.

In order to determine the user compliance, a piecewise function may be used.

A minimum TIB (e.g., 0 hours) may be set, and in order to determine the user compliance based on a piecewise function, a maximum TIB may be set. The maximum TIB is the maximum bedtime for which a person can sleep, for example, 20 hours.

The maximum TIB duration may be changed based on a defined minimum value (e.g., ten hours), and may be adjusted based on sleeping data of the user. Alternatively, the maximum TIB duration may be set to be greater than at least 2×R-TIB.

When the R-TIB is a and the maximum TIB duration is b, the user compliance curve may be determined based on the following equation.

$$y = \{ x < a : - \frac{I 00}{{a}^{2}} {( x - a )}^{2} + 1 \; 00 , x \geq a : - \frac{100}{{( b - a )}^{2}} {( x - a )}^{2} + 100 \} \qquad [ Equation 1 ‐ 2 ]$$

Here, x denotes a TIB and y denotes user compliance. For example, when the R-TIB is 6 and the TIB is 4, the user compliance is 88.9%, and when the R-TIB is 6 and the TIB is 9, the user compliance is 97.9%.

The user compliance curve may be defined as a power of 2 as shown in Equation 1-2, or may be defined as a power of another even number providing a steeper curve. The number for an exponent for determining the user compliance curve may be adaptively changed according to the user compliance feedback value.

Referring to the user compliance curve, the R-TIB is generally less than half of the maximum TIB duration. Accordingly, the user compliance curve may be defined to determine the user compliance more strictly in a part having a sleep time less than the R-TIB compared to a part having a sleep time more than the R-TIB.

However, when the R-TIB is greater than half of the maximum TIB duration (e.g., R-TIB=6 and max TIB duration=10), the user compliance curve may have a steep slope such that the compliance of people who slept more than the R-TIB decreases relatively rapidly, as shown on the right side of the curve.

The user compliance curve may be generated based on the fact that sleeping less is less desirable than sleeping more in the treatment of insomnia.

The user compliance determiner 120 integrates data from the decision tree algorithm to classify compliance behavior. Low compliance may trigger an intervention through temperature control adjustments to reinforce bedtime adherence and/or haptic stimulation to encourage wakefulness at the second R-TIB. The decision tree algorithm adapts future R-TIB recommendations based on past compliance trends.

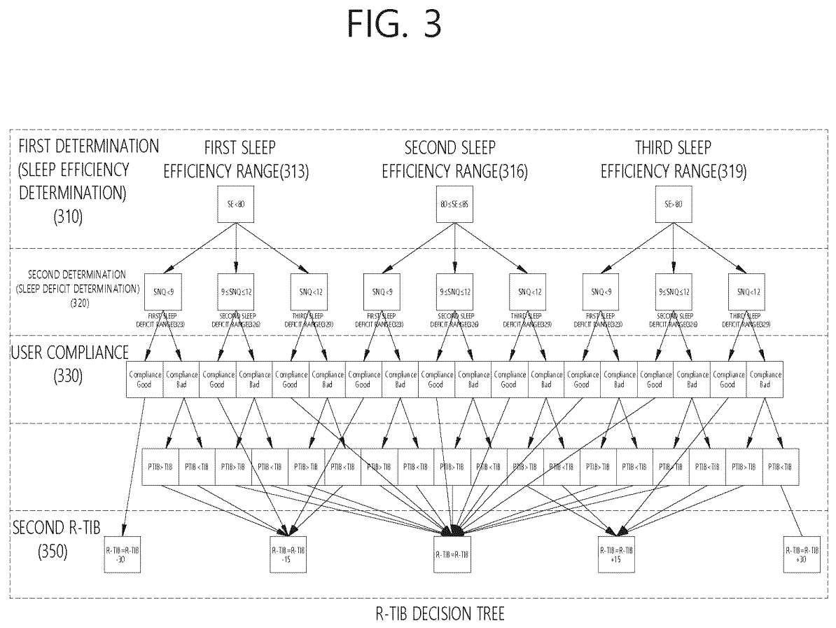

FIG. 3 is a conceptual diagram illustrating an operation of an R-TIB determiner according to an embodiment of the present invention.

In FIG. 3, a method of the R-TIB determiner 130 determining an R-TIB is disclosed.

Referring to FIG. 3, a method of the R-TIB determiner 130 determining an R-TIB by performing a first determination (a sleep efficiency determination) 310 on the sleep of the user and performing a second determination (a sleep deficit determination) 320 on the sleep of the user is disclosed.

Among three sleep efficiency ranges set based on the first determination (the sleep efficiency determination) 310, one sleep efficiency range may be set as the sleep efficiency range of the user. Among three sleep deficit ranges set based on the second determination (the sleep deficit determination) 320, one sleep deficit range may be set as the sleep deficit range of the user.

The interval and the number of the range sections may be varied, and a method of varying the interval and the number of the range sections may also be included in the scope of the present invention. In addition, a first sleep efficiency range 313, a second sleep efficiency range 316, and a third sleep efficiency range 319, which are three sleep efficiency ranges set based on the first determination (the sleep efficiency determination) 310 in the present invention, and a first sleep deficit range 323, a second sleep deficit range 326, and a third sleep deficit range 329, which are three sleep deficit ranges set based on the second determination (the sleep deficit determination) 320 in the present invention are arbitrary and subject to change.

First, the first determination (the sleep efficiency determination) 310 may be performed on the sleep of the user.

The sleep efficiency of the user may be classified based on sleep efficiency (SE) determined by the sleep quality determiner 100, as one of the first sleep efficiency range (SE<80) 313, the second sleep efficiency range (80<=SE<=85) 316, and the third sleep efficiency range (SE>85) 319.

After the determination of the sleep efficiency range, the second determination (the sleep deficit determination) 320 may be performed on the sleep of the user.

The sleep deficit of the user may be classified based on a sleep need questionnaire (SNQ) determined by the sleep quality determiner 100 as one of the first sleep deficit range (SNQ<9) 323, the second sleep deficit range (9<=SNQ<=12) 326, and the third sleep deficit range (SNQ>12) 329.

After the first determination (the sleep efficiency determination) 310 and the second determination (the sleep deficit determination) 320, user compliance 330 may be determined. The user compliance 330 may be determined through a method of determining user compliance 330 based on the first R-TIB previously recommended to the user and the actual TIB of the user described above in FIG. 2.

When the user compliance 330 is greater than or equal to a threshold, the user compliance 330 may be determined to be in a first state (compliance good), and when the user compliance 330 is less than the threshold value, the user compliance 330 may be determined to be in a second state (compliance bad).

In response to the user compliance 330 being in the second state, a comparison in size between the first R-TIB and the TIB may be performed. It may be determined whether the user compliance 330 is in the second state because the first R-TIB is larger than the TIB, and conversely, whether the user compliance 330 is in the second state because the first R-TIB is smaller than the TIB.

A second R-TIB 350 may be determined.

The second R-TIB 350 may be determined based on the user sleep efficiency range, the user sleep deficit range, the user compliance, and the difference in size between the first R-TIB and the TIB.

Considering the sleep quality, the sleep time, and the first R-TIB and the TIB, option 1) determining the second R-TIB by decreasing the first R-TIB, option 2) determining the second R-TIB by maintaining the existing first R-TIB, or option 3) determining the second R-TIB by decreasing the first R-TIB may be determined. In addition, an increase amount of the first R-TIB and a decrease amount of the first R-TIB may also be determined in consideration of the sleep quality, the sleep time, the first R-TIB, and the TIB.

In FIG. 3, a rule for determining the second R-TIB in consideration of the sleep quality, the sleep time, the first R-TIB, and the TIB is illustrated.

Basically, the existing first R-TIB may be set to be maintained when the sleep efficiency is relatively high, the sleep deficit is relatively low, and the user compliance is relatively high, and adjustment of the first R-TIB may be performed in consideration of the sleep efficiency and the sleep deficit. Specifically, the adjustment of the first R-TIB may be set to decrease the first R-TIB as the sleep efficiency is lower, and may be set to increase the first R-TIB as the sleep efficiency is higher. In addition, the adjustment of the first R-TIB may be set to decrease the first R-TIB as the sleep deficit is relatively small, and may be set to increase the first R-TIB as the sleep deficit is relatively large.

The processor (140) actively transmits a control signal to at least one predetermined supplemental device, including a temperature controller configured to adjust the ambient sleep environment based on second R-TIB data and a haptic actuator configured to provide stimulation to wake the user at the recommended second R-TIB.

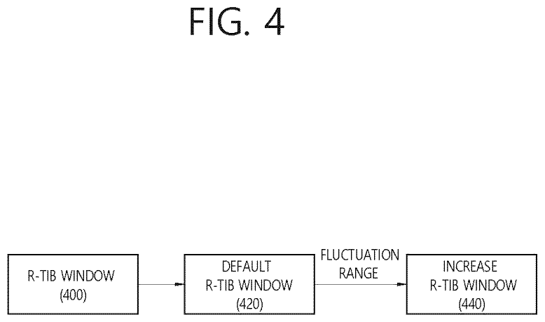

FIG. 4 is a conceptual diagram illustrating a method of extracting data for determining an R-TIB according to an embodiment of the present invention.

In FIG. 4, a method of setting an R-TIB window to determine an R-TIB with high accuracy and reliability is disclosed.

Referring to FIG. 4, an R-TIB window 400 may be basically set based on sleep data of the most recent n days (e.g., n=5), which is unmodifiable.

In order to increase the user's autonomy, sleep data of three days including the current date may be set to be directly edited and written by the user. Accordingly, the R-TIB window 400 may be basically determined based on sleep data of the most recent n days, which is unmodifiable. Such a setting of the R-TIB window 400 obviates a need to consider changes such as a case in which the sleep diary written by the user is modified and thus the R-TIB is repeatedly applied, so that the reliability of the R-TIB may be increased.

In order to select the R-TIB window 400, a fluctuation range may be checked. For example, there may be a large difference between a weekend sleep pattern and a weekday sleep pattern. In the case of weekends, the fluctuation range of sleep may be relatively large. When exceptional data is included as an outlier in the calculation of the R-TIB, a result that is inaccurate or unhelpful to the patient may be output.

The R-TIB window 400 may be initially set to a default R-TIB window (e.g., five days) 420, and a fluctuation range of the default R-TIB window 420 may be determined. The fluctuation range of the default R-TIB window 420 may be calculated based on a standard deviation. When the fluctuation range of the default R-TIB window 420 is greater than or equal to a threshold fluctuation range, an increase R-TIB window 440 of six days may be set by adding one more day to the original default R-TIB window 420. When the increase R-TIB window 440 is set, the fluctuation range may decrease as more is added to the sleep diary, and the R-TIB may be determined from sleep data of n′ pieces of sleep data of the user included in the increase R-TIB window 400 based on the fluctuation range being less than the threshold fluctuation range. The R-TIB determined through the method of adjusting the R-TIB window may have improved reliability and accuracy.

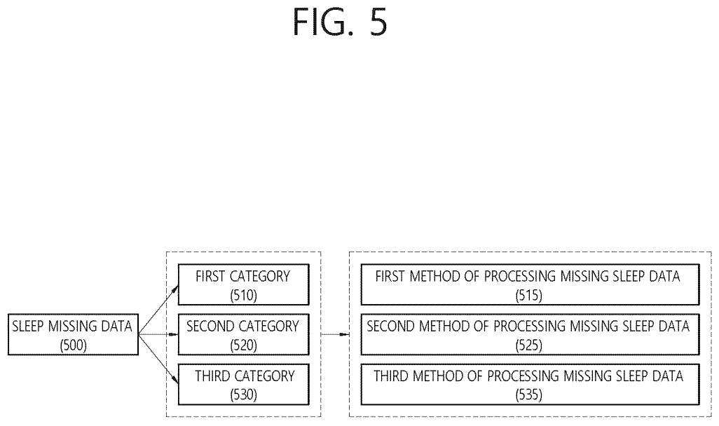

FIG. 5 is a conceptual diagram illustrating a method of processing missing data according to an embodiment of the present invention.

In FIG. 5, a method of determining an R-TIB through processing of missing sleep data that is missing in sleep data is disclosed.

Referring to FIG. 5, missing sleep data 500 may be classified into three categories.

A first category 510 is a case in which missing sleep data 500 is generated without a miss pattern.

A second category 520 is a case in which missing sleep data 500 is generated with a miss pattern.

A miss pattern is a regular occurrence of omission of sleep data, such as omission of sleep data at a specific time. For example, when missing sleep data is generated because sleep data is not written on the weekend, the missing sleep data is considered as having a miss pattern.

A third category 530 is a case in which missing sleep data 500 is generated with an intention to omit sleep data. For example, this is a case of omission of sleep data generation on a day when the user fails to sleep, to intentionally hide that the user fails to sleep.

In the present invention, the missing sleep data 500 may be processed in consideration of the three categories of missing sleep data in various ways to determine the R-TIB.

As a first method 515 of processing missing sleep data, a likewise deletion method may be used. The likewise deletion method is a method of determining the R-TIB by excluding ungenerated sleep data regardless of the category.

A second method 525 of processing missing sleep data is a method of determining the R-TIB by excluding missing sleep data 500 corresponding to the second category 520, and estimating missing sleep data 500 corresponding to the first category 510 and the third category 530 to generate sleep data. The missing sleep data 500 of the first category 510 may be generated through estimation based on sleep data of nearby dates, that is, sleep data of a previous date and a following date. The missing sleep data 500 of the third category 530 may be generated through recommendation based on sleep data of a date having the lowest sleep efficiency and the highest sleep deficit in the R-TIB window.

A third method 535 of processing missing sleep data is a method of determining the R-TIB by excluding missing sleep data 500 corresponding to the first category 510 and the second category 520, and by estimating only missing sleep data corresponding to the third category 530 to generate sleep data.

The first method 515 of processing missing sleep data, the second method 525 of processing missing sleep data, and the third method 535 of processing missing sleep data may be selectively used according to a user.

In consideration of the existing user compliance of the user, the first method 515 of processing missing sleep data may be used when the user compliance is greater than or equal to a first threshold value.

In consideration of the existing user compliance of the user, the second method 525 of processing missing sleep data may be used when the user compliance is greater than or equal to a second threshold value and less than the first threshold value.

In consideration of the existing user compliance of the user, the third method 535 of processing missing sleep data may be used when the user compliance is less than the second threshold value.

That is, considering whether the existing user has complied with the method of treating a sleep disorder based on data according to the embodiment of the present invention, the R-TIB may be determined based on a stricter criterion as the user compliance is lower.

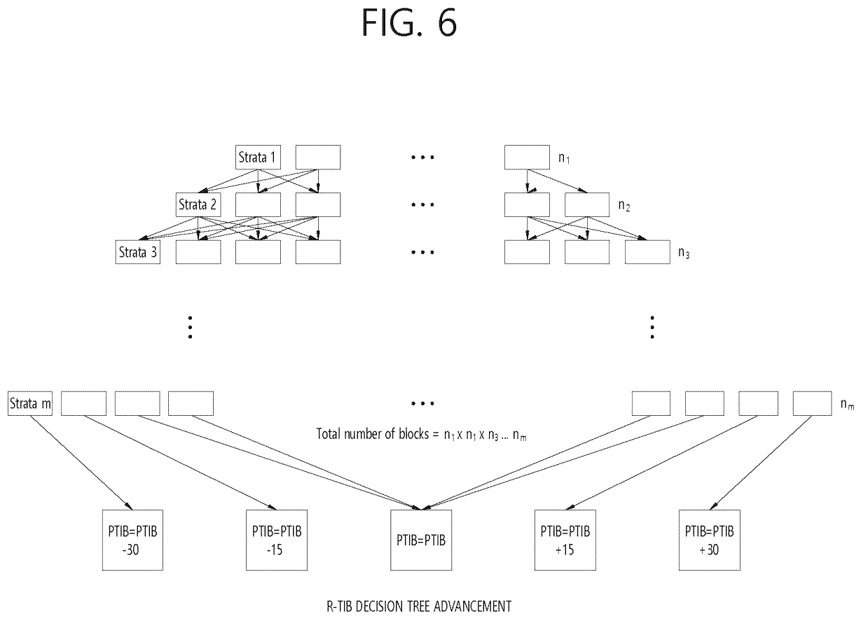

FIG. 6 is a conceptual diagram illustrating a method of determining an R-TIB according to an embodiment of the present invention.

In FIG. 6, a method of advancing the determination of the R-TIB by further specifying sleep efficiency ranges and sleep deficit ranges is illustrated.

Referring to FIG. 6, the sleep efficiency ranges and the sleep deficit ranges in the R-TIB determination method disclosed in FIG. 3 are divided in more detail so that an R-TIB decision tree may be advanced.

Through an increase in the number of sleep efficiency range sections, in which sleep efficiency range sections are divided into a larger number of sections, and/or an increase in the number of sleep deficit range sections, in which sleep deficit range sections are divided into a larger number of sections, the R-TIB may be determined through a more detailed classification.

The R-TIB may be determined based on a stricter criterion as user compliance is lower. Therefore, as the user compliance is lower, the R-TIB may be more precisely determined through an increase in the number of the sleep efficiency range sections and an increase in the number of the sleep deficit range sections. Conversely, as the user compliance is higher, the R-TIB may be determined through a decrease in the sleep efficiency range sections and a decrease in the number of the sleep deficit range sections.

The embodiments of the present invention can be implemented in the form of program commands executable by a variety of computer components and may be recorded on a computer readable medium. The computer readable medium may include, alone or in combination, program commands, data files and data structures. The program commands recorded on the computer readable medium may be components specially designed for the present invention or may be usable to a skilled person in the field of computer software. Computer readable record media include magnetic media such as a hard disk, a floppy disk, or a magnetic tape, optical media such as a compact disc read only memory (CD-ROM) or a digital video disc (DVD), magneto-optical media such as floptical disks, and hardware devices such as a ROM, a random-access memory (RAM), or a flash memory specially designed to store and carry out programs. The program commands include not only a machine language code made by a compiler but also a high-level code that can be used by an interpreter etc., which is executed by a computer. The hardware device may be configured to act as one or more software modules in order to perform the operations of the present invention, or vice versa.

As is apparent from the above, a sleep disorder of a user can be treated by recommending a more therapeutically effective bedtime in consideration of the sleep efficiency of the user using data.

A sleep disorder of a user can be treated by appropriately adjusting a recommended bedtime of the user to control the balance between the patient's compliance and therapeutic performance.

A sleep disorder of a user can be treated through adaptive management of sleep data and generation of an R-TIB tree in consideration of the user compliance of the user.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

The effects of the present application are not limited to the above-described effects, and effects not described may be clearly understood by those of ordinary skill in the art from the present specification and the accompanying drawings.

While the invention has been shown and described with respect to particulars, such as specific components, embodiments, and drawings, the embodiments are used to aid in the understanding of the present invention rather than limiting the present invention, and those skilled in the art should appreciate that various changes and modifications are possible without departing from the spirit and scope of the invention.

Therefore, the spirit of the present invention is not defined by the above embodiments but by the appended claims of the present invention, and the scope of the present invention is to cover not only the following claims but also all modifications and equivalents derived from the claims.

What is claimed is:

1. A sleep disorder treatment apparatus in the form of a device worn by a user for performing sleep disorder treatment on the user based on data using a decision tree algorithm, the sleep disorder treatment apparatus comprising a processor and one or more memory devices communicatively coupled to the processor, and the one or more memory devices stores instructions operable when executed by the processor to perform the steps of:
determining actual sleep time in bed (TIB) based at least on objective evaluation data received by at least one sensor of the wearable device, the at least one objective evaluation data including at least one of respiration data and brain waves data;
determining sleep quality including sleep efficiency and sleep deficit using the decision tree algorithm and objective evaluation data received by the at least one sensor of the wearable device, the objective evaluation data including at least one of respiration data and brain waves data;
setting a recommended time in bed (R-TIB) window, wherein the R-TIB window is initially set to a default R-TIB window, and a fluctuation range of the default R-TIB window is calculated based on a standard deviation, and wherein when the fluctuation range of the default R-TIB window is greater than or equal to a threshold fluctuation range, the R-TIB window is increased by adding one more day to the default R-TIB window, and wherein the R-TIB window is determined based on unmodifiable sleep data of the most recent n days;
processing missing sleep data;
determining a first recommended time in bed (R-TIB) based on the sleep efficiency, the sleep deficit, the R-TIB window and the processed missing sleep data; and
determining a second R-TIB, using the decision tree algorithm, on the basis of the sleep quality, the processed missing sleep data, and user compliance; and
determining user compliance with the first R-TIB, using the decision tree algorithm, based on slopes that are set to be different according to ranges of the TIB or determined based on a user compliance curve using a piecewise function including a minimum TIB, a maximum TIB and the first R-TIB which is less than one half of the maximum TIB, the user compliance curve being parabolic,
wherein the first R-TIB is a sleep time recommended to a user earlier, and
the second R-TIB is a sleep time recommended to the user later, and
wherein the second R-TIB is determined based on a user sleep efficiency range, a user sleep deficit range, the user compliance, and a difference in size between the first R-TIB and the TIB,
wherein the missing sleep data is classified into one of three categories: a first category in which the missing sleep data is generated without a miss pattern, a second category in which the missing sleep data is generated with a miss pattern, and a third category in which the missing sleep data is intentionally hidden when the user failed to sleep on a particular day,
wherein the sleep disorder treatment apparatus further comprises at least one predetermined supplemental device, and the processor transmits a control signal to the at least one predetermined supplemental device based on the second R-TIB and the instruction stored in the one or more memory devices, and
wherein the processor, based on the instruction stored in the one or more memory devices, actively controls the at least one predetermined supplemental device including a temperature controller and a haptic actuator, wherein:
the temperature controller is configured to induce sleep by adjusting the temperature to the predetermined temperature to maintain an optimal sleep environment based on the second R-TIB; and
the haptic actuator is configured to wake the user by providing the predetermined stimulation based on the second R-TIB; and
the processor, based on the instruction stored in the one or more memory devices, continuously monitors the user's sleep state and dynamically adjusts treatment in real-time based on feedback from the wearable sensors.

2. The sleep disorder treatment apparatus of claim 1,
wherein the user sleep efficiency range is a sleep efficiency range of the user among “a”, wherein (here, “a” is a natural number) set sleep efficiency ranges based on a first determination on the sleep efficiency,
wherein the user sleep deficit range is a user sleep deficit range of the user among “b”, wherein (here, “b” is a natural number set sleep deficit ranges based on a second determination on the sleep deficit, and
wherein the user sleep deficit range is determined, after determination of the user sleep efficiency range, based on the determined user sleep efficiency range.

3. The sleep disorder treatment apparatus of claim 1, wherein the decision tree algorithm includes performing a first determination of the sleep efficiency by the sleep quality determiner, a second determination of the sleep deficit by the sleep quality determiner, a third determination of the user compliance by the user compliance determiner, and a fourth determination of the second R-TIB by the R-TIB determiner,
wherein the second R-TIB is applied to real-time control of the temperature controller and the haptic actuator.

4. The sleep disorder treatment apparatus of claim 3, wherein the sleep efficiency is classified as one of the sleep efficiency ranges, and wherein the sleep deficit is classified based on a sleep need questionnaire, and wherein the user compliance is determined based on the first R-TIB and the TIB.

5. The sleep disorder treatment apparatus of claim 1, wherein the missing sleep data is processed by excluding ungenerated sleep data regardless of the categories when the user compliance is greater than or equal to a first threshold value.

6. The sleep disorder treatment apparatus of claim 5, wherein the missing sleep data is processed, when the user compliance is greater than or equal to a second threshold value and less than the first threshold value, by excluding ungenerated sleep data corresponding to the second category and estimating the missing sleep data corresponding to the first category and the third category.

7. The sleep disorder treatment apparatus of claim 6, wherein the missing sleep data is processed, when the user compliance is less than the second threshold value, by excluding ungenerated sleep data corresponding to the first category and the second category, and by estimating only missing sleep data corresponding to the third category.

8. The sleep disorder treatment apparatus of claim 1, wherein the TIB is determined based on subjective evaluation data and/or objective evaluation data including respiration, brain waves or movement.

9. A sleep disorder treatment method using a device worn by a user for performing sleep disorder treatment on the user based on data using a decision tree algorithm, wherein the device comprises a processor and one or more memory devices communicatively coupled to the processor, and the one or more memory devices stores instructions operable when executed by the processor to perform the steps of:
determining actual sleep time in bed (TIB) based at least on objective evaluation data received by at least one sensor of the wearable device, the at least one objective evaluation data including at least one of respiration data and brain waves data;
determining sleep quality including sleep efficiency and sleep deficit using the decision tree algorithm and objective evaluation data received by the at least one sensor of the wearable device, the objective evaluation data including at least one of respiration data and brain waves data;
setting a recommended time in bed (R-TIB) window, wherein the R-TIB window is initially set to a default R-TIB window, and a fluctuation range of the default R-TIB window is calculated based on a standard deviation, and wherein when the fluctuation range of the default R-TIB window is greater than or equal to a threshold fluctuation range, the R-TIB window is increased by adding one more day to the default R-TIB window, and wherein the R-TIB window is determined based on unmodifiable sleep data of the most recent n days;
processing missing sleep data;
determining a first recommended time in bed (R-TIB) based on the sleep efficiency, the sleep deficit, the R-TIB window and the processed missing sleep data; and
determining a second R-TIB, using the decision tree algorithm, on the basis of the sleep quality, the processed missing sleep data, and user compliance; and
determining user compliance with the first R-TIB, using the decision tree algorithm, based on slopes that are set to be different according to ranges of the TIB or determined based on a user compliance curve using a piecewise function including a minimum TIB, a maximum TIB and the first R-TIB which is less than one half of the maximum TIB, the user compliance curve being parabolic,
wherein the first R-TIB is a sleep time recommended to a user earlier, and
the second R-TIB is a sleep time recommended to the user later, and
wherein the second R-TIB is determined based on a user sleep efficiency range, a user sleep deficit range, the user compliance, and a difference in size between the first R-TIB and the TIB,
wherein the missing sleep data is classified into one of three categories: a first category in which the missing sleep data is generated without a miss pattern, a second category in which the missing sleep data is generated with a miss pattern, and a third category in which the missing sleep data is intentionally hidden when the user failed to sleep on a particular day,
wherein the device further comprises at least one predetermined supplemental device, and the processor transmits a control signal to the at least one predetermined supplemental device based on the second R-TIB and the instruction stored in the one or more memory devices, and
wherein the processor, based on the instruction stored in the one or more memory devices, actively controls the at least one predetermined supplemental device including a temperature controller and a haptic actuator, wherein:
the temperature controller is configured to induce sleep by adjusting the temperature to the predetermined temperature to maintain an optimal sleep environment based on the second R-TIB; and
the haptic actuator is configured to wake the user by providing the predetermined stimulation based on the second R-TIB; and
the processor, based on the instruction stored in the one or more memory devices, continuously monitors the user's sleep state and dynamically adjusts treatment in real-time based on feedback from the wearable sensors.

10. The method of claim 9,
wherein the user sleep efficiency range is a sleep efficiency range of the user among “a”, wherein (here, “a” is a natural number) set sleep efficiency ranges based on a first determination on the sleep efficiency,
wherein the user sleep deficit range is a user sleep deficit range of the user among “b”, wherein (here, “b” is a natural number set sleep deficit ranges based on a second determination on the sleep deficit, and
wherein the user sleep deficit range is determined, after determination of the user sleep efficiency range, based on the determined user sleep efficiency range.

11. The method of claim 9, wherein the decision tree algorithm includes performing a first determination of the sleep efficiency by the sleep quality determiner, a second determination of the sleep deficit by the sleep quality determiner, a third determination of the user compliance by the user compliance determiner, and a fourth determination of the second R-TIB by the R-TIB determiner,
wherein the second R-TIB is applied to real-time control of the temperature controller and the haptic actuator.

12. The method of claim 9, wherein the sleep efficiency is classified as one of the sleep efficiency ranges, and wherein the sleep deficit is classified based on a sleep need questionnaire, and wherein the user compliance is determined based on the first R-TIB and the TIB.

13. The method of claim 9, wherein the missing sleep data is processed by excluding ungenerated sleep data regardless of the categories when the user compliance is greater than or equal to a first threshold value.

14. The method of claim 13, wherein the missing sleep data is processed, when the user compliance is greater than or equal to a second threshold value and less than the first threshold value, by excluding ungenerated sleep data corresponding to the second category and estimating the missing sleep data corresponding to the first category and the third category.

15. The method of claim 14, wherein the missing sleep data is processed, when the user compliance is less than the second threshold value, by excluding ungenerated sleep data corresponding to the first category and the second category, and by estimating only missing sleep data corresponding to the third category.

16. The method of claim 9, wherein the TIB is determined based on subjective evaluation data and/or objective evaluation data including respiration, brain waves or movement.
